(19) United States
(12) Patent Application Publication
(10) Pub. No.: US 2021/0378346 A1
(43) Pub. Date: Dec. 9, 2021

(54) INVISIBLE FACE MASK

(71) Applicant: Harry Abghari, San Juan Capistrano, CA (US)

(72) Inventor: Harry Abghari, San Juan Capistrano, CA (US)

(21) Appl. No.: 16/942,035

(22) Filed: Jul. 29, 2020

Related U.S. Application Data
(60) Provisional application No. 63/033,996, filed on Jun. 3, 2020.

(51) Int. Cl. A42B 3/28; A41D 13/11; A62B 23/02; A61F 9/04

(52) U.S. Cl. CPC A42B 3/286; A41D 13/1184; A42B 3/30; A61F 9/045; A62B 23/02

(57) ABSTRACT

The inventive Invisible Face Mask apparatus comprises a headgear or headwear such as a cap, helmet, strap or a visor, which is equipped with an air purifying mechanism, such as an air filter, that filters ambient air, and an air blower which generates an airflow pushing the purified air in a downward direction in front of and adjacent to the user's face.

### CROSS-REFERENCE TO RELATED PATENT APPLICATIONS

Embodiments of the present invention relate to U.S. Provisional Application Ser. No. 63/033,996, filed on Jun. 3, 2020, entitled “INVISIBLE FACE MASK”, the contents of which are incorporated by reference herein in its entirety and which is a basis for a claim of priority.

### FIELD OF THE INVENTION

Embodiments of the present disclosure generally relate to a system and apparatus for preventing or limiting entry of harmful germs and viruses such as the viruses associated with causing infectious diseases such as the novel coronavirus (COVID-19), the seasonal flu, and the common cold, as well as disease causing bacteria and funguses, into people's respiratory systems.

### BACKGROUND OF THE INVENTION

Studies have confirmed that the COVID-19 virus spreads through respiratory droplets between people who are in proximity closed to one another through talking or just exhaling by infected persons. The pandemic has thus refocused the world on overcoming the challenges of controlling and limiting air-bone spread of respiratory infectious diseases. In that effort, mitigation measures have been developed and implemented, which, in varying degrees, have been proven effective in slowing down the spread of COVID-19, while efforts to develop effective cures and vaccines are under way.

Currently deployed COVID-19 mitigation measures include quarantining for those who have been confirmed as having caught the virus, encouraging personal hygiene, requiring social distancing and, more recently, mandatory use of face masks. Of these measures, mandating of masks has been the most controversial as it has been variously labeled as a symbol of fear and having a negative impact on social relations and norms of discourse; at the same time when the effectiveness of conventional masks has been questioned.

Protection provided by conventional cloth face masks suffers from practical drawbacks. Conventional cloth masks worn by vast majority of the users are not very effective in preventing airborne germs and viruses from entering people's respiratory system. Even N95, touted as the most effective commercially available mask, is only capable of filtering out 95% of the smallest particles in the air; even then, the mask has to be worn properly and fit just right in order to work. Much more commonly used are cloth masks, also known as surgical masks, that do not require a tight facial fit. Such masks, although effective in limiting the reach of droplets exhaled from the user, are relatively porous and not tight enough to block passage of all germs. Importantly, conventional cloth masks are ineffective in blocking the smaller particles that carry the coronavirus.

Another disadvantage of conventional cloth masks is the chronic challenge of maintaining their proper fit and consistent use among certain age and demographic groups, such as the very young, the handicapped and one of the most COVID-19 vulnerable groups, the very old.

Yet another disadvantage of use of conventional cloth face masks is that they frequently exasperate breathing difficulties certain individuals suffer from. Recent literature in medical publications have noted that use of conventional face masks may interfere with normal levels of oxygen intake and result in increased intake of carbon dioxide exhaled during breathing. There are reported cases of those wearing masks becoming disoriented or even passing out due to the oxygen/carbon dioxide balance. This condition can be especially acute for individuals suffering from certain pre-existing conditions, such as asthma, other lung conditions or anxiety.

Even willing users of conventional cloth masks will face a special challenge during the hot summer months, occasions of high humidity in the air and during periods of moderately intensive activity, when conventional cloth masks may make breathing more cumbersome.

Social distancing is another component of mitigation protocol. Under current practice, social distancing of six feet is recommended with use of conventional masks to keep COVID-19 virus carrying droplets exhaled during talking or breathing from reaching others. That distance, although necessary, is economically damaging and unfeasible as it severely limits attendance at public places including bars, restaurant, movie theatres, sports stadiums, shopping centers, and other public places. A more natural and reasonable distance of three feet will, by itself, quadruple the number of patrons who can safely attend such places, making three feet distancing much more realistic and workable.

There have been a numeral of attempts to create an air flow-based barrier to in-breathing of air borne particulates. However, these efforts suffer from deficiencies when dealing with the problem they seek to address.

U.S. Patent Application Publication No. 2013/0118506, entitled “Method for protecting breathing organs and eyes from aerosols and device for implementing the same,” discloses an air flow apparatus, which uses ambient air with no filtration mechanism. Moreover, the disclosed air flow volume in the abandoned application would require large batteries and would be too bulky and noisy to be of practical everyday use.

U.S. Pat. No. 7,823,586, entitled “Personal respiratory protection system” discloses a “rigid” system, not designed for popular use, but for use limited to airplane rides, doctor and hospital visits. Another disadvantage of the disclosed system is its use of an ultraviolet light disinfecting mechanism, which, in itself raises a potential health issues for certain users.

U.S. Patent Application Publication No. 2003/0188743, entitled “Air Curtain Device” discloses an apparatus including a headset with a fan mounted on it. Among the disadvantages of the disclosed system is the sideways direction of air flow, which will spread the air born virus from infected person wearing the device to others nearby. Moreover, the devise is difficult to wear and will fall out of its adjusted position in response to user's motions and does not filter ambient air.

U.S. Patent Application Publication No. 2018/0021607, entitled “Hat and Air Filtration System” discloses an air blowing system, which includes a rigid hardhat and thus not meant for common usage. The system depicted directs air flow through the bottom section of the hardhat, which results in air leakage through gaps between headgear and user's head, thus hindering effective downward flow across user's face.

U.S. Pat. No. 10,653,197 entitled “Hard hat with filtered, battery-operated air flow system and method” also discloses a hard hat system, which is not practical for common, everyday use and additionally includes an external backpack and does not filter ambient air. Moreover, the disclosed airflow system is ineffective in covering user's face as the air flow is angled away from user's face.

JP2009061233, entitled “Fresh air feeding device to face” provides a mechanism which it states is designed to protect user against polluted air. The disclose system suffers from the disadvantage of not being very effective against viruses as the it includes a gap of about 3″ this dimension is per scaling the average size of hat of the disclosure system and location of air nasal as shown in photos) between user's face and the air flow path, which results in inflow of unfiltered infectious air, pulled in by turbulence of discharge air stream, towards user's face. The other disadvantage is that all system fan, battery, control etc. installed over the brim of hat with approximately same length of hat, this makes it impractical to be worn in regular day activity.

### SUMMARY OF THE DISCLOSURE

Various embodiments of the present disclosure are directed to an Invisible Face Mask (IFM) comprising a headgear or headwear such as a cap, helmet or a strap, which is equipped with an air purifying mechanism, such as an air filter, that filters ambient air, and an air blower which generates an airflow pushing the purified air in a downward direction in front of and adjacent to the user's face. The inventive system and apparatus of the Invisible Face Mask acts to make purified air available to the user for breathing, and, at the same time shields the user from inhaling harmful airborne particulates that carry the COVID-19 and other infectious germs, and additionally directs any harmful germs and viruses exhaled by the user during breathing, coughing or sneezing, towards the ground and away from others who are in the vicinity of the user.

### DETAILED DESCRIPTION OF PREFERRED EMBODIMENTS

The following description is not to be interpreted or applied in a limiting sense, but as illustrating the general principles and aspects of the invention, since the scope of the invention is best defined by the following claims. Various inventive features are described below that can each be used independently of one another or in combination with other disclosed and undisclosed features.

FIG. 1 shows an embodiment of the present disclosure. Reference numeral 101 shows a conventional popularly used headwear cap worn by user 100. Ambient air 102 is taken in through air filter 103, preferably with a 17 GPM to 30 GPM (Variable or 3 Speed switch) centrifugal Aair fan with an HEPA filter, which is preferably part of a battery-operated air purifying system 104. Preferably, the battery is rechargeable. Purified Air passes through air hose 105 and is discharged through nozzle 106, preferably having a discharge surface of 2 square inches, in front of and in a direction 107 substantially parallel to user's face, preferably at a speed of 38 inches per second which can be increased to 76 inches per second, thus preventing infected ambient air 102 to be breathed in by user 100. Preferably, the air nozzle is configured to be adjustable. In one embodiment, the inventive apparatus is configured to weigh about one pound.

In one embodiment, a control system for the air blower has a battery level indicator to show the an approximately remaining charge of battery.

FIG. 2 shows a front view of the embodiment shown in FIG. 1. Ambient air 102 is taken in through the apparatus 111 mounted on cap 101. Filtered air 107 flows across and parallel to user's face in a curtain like fashion, protecting user from breathing in harmful germs and viruses.

FIG. 3 shows another embodiment of the present disclosure. Reference numeral 109 shows a conventional widely used bike helmet worn by user 100. Ambient air 102 is taken in through air filter 103, which is part of a battery-operated air purifying system 104. Purified Aair passes through air hose 105 and is discharged through nozzle 106, which is preferably located right over user's forehead to allow air discharge flow in front of and in a direction 107 substantially parallel to user's face, thus preventing infected ambient air 102 to be breathed in by user 100. Preferably, the air flow speed is between approximately 38 and 76 inches per second.

Advantageously, the inventive IFM of the present disclosure can be worn in lieu of conventional face masks in locations of public gathering, such as restaurants, sports stadiums, movie theaters, hotels, concerts, museums, parks, airplanes, and any place where significant number of people are in attendance.

In another embodiment, the disclosed air fan is adjustable to adjust the air flow according to the preference of the user and the environment and circumstance in which the IFM is used. Preferably the inventive system is configured to be monitored/control through a mobile phone application with the user mobile phone. the air purifier comprises a replaceable H13, HEPA filter and (Optional) an activated carbon filter. (Activated carbon filter removes chlorine, particles such as sediment, volatile organic compounds (VOCs), taste and odor. Preferably, the air purifier is capable of filtering ambient air to 99.97% purity and of filtering particles down to a diameter of 0.3 micron. the entire fan, & a battery, and a control system are mounted over a side, back or top of a hat or sun visors and an air hose is included used to transfer air to a front of the user's face. Preferably, the air purifier and the air blower are configured to be mounted as a kit over the headgear or headwear. In one embodiment, the inventive IMF includes a short face shield to protect the user in harsh environments.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above-described embodiment, method, and examples, but by all embodiments and methods within the scope and spirit of the invention as claimed.

### BRIEF DESCRIPTION OF THE DRAWINGS

FIG. 1 is a schematic diagram according to an exemplary embodiment of the present disclosure.

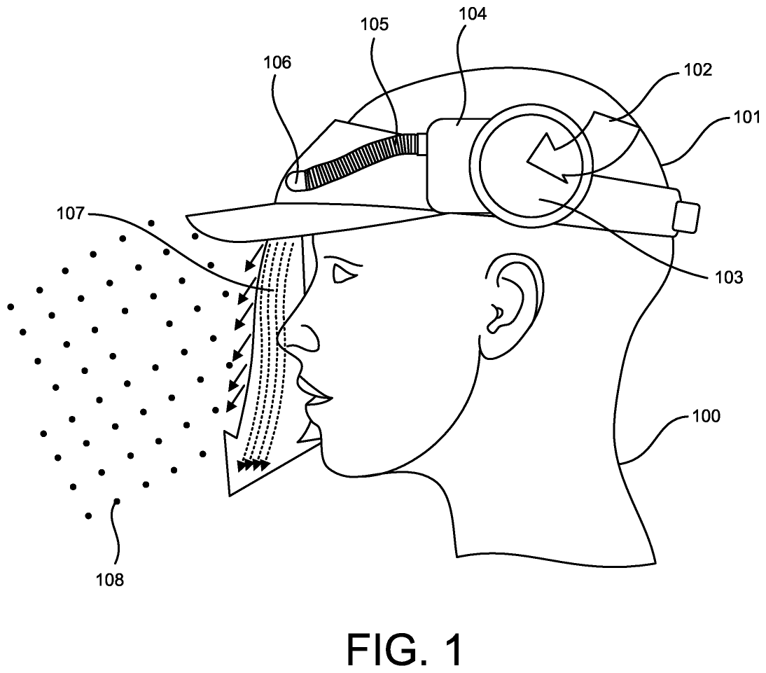

FIG. 2 is a schematic diagram according to an exemplary embodiment of the present disclosure.

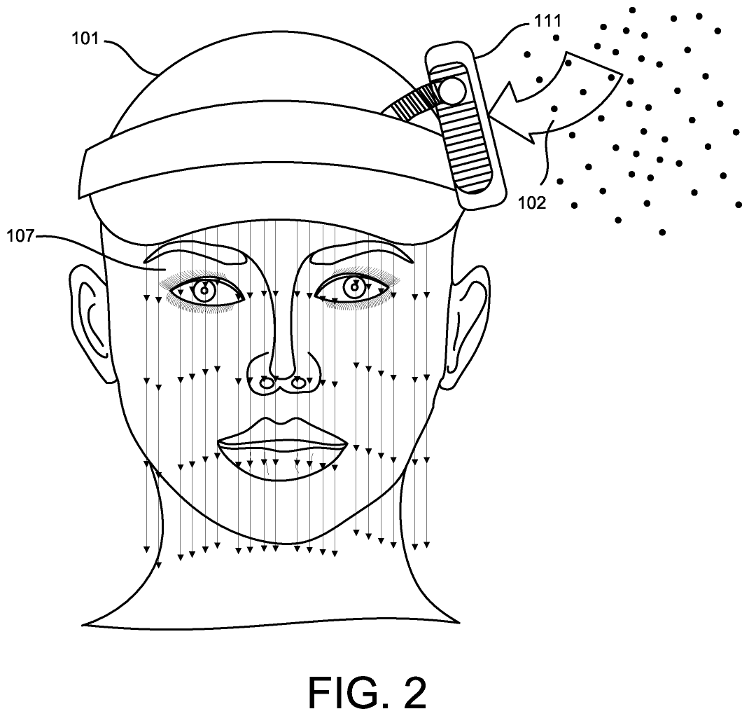

FIG. 3 is a schematic diagram according to an exemplary embodiment of the present disclosure.

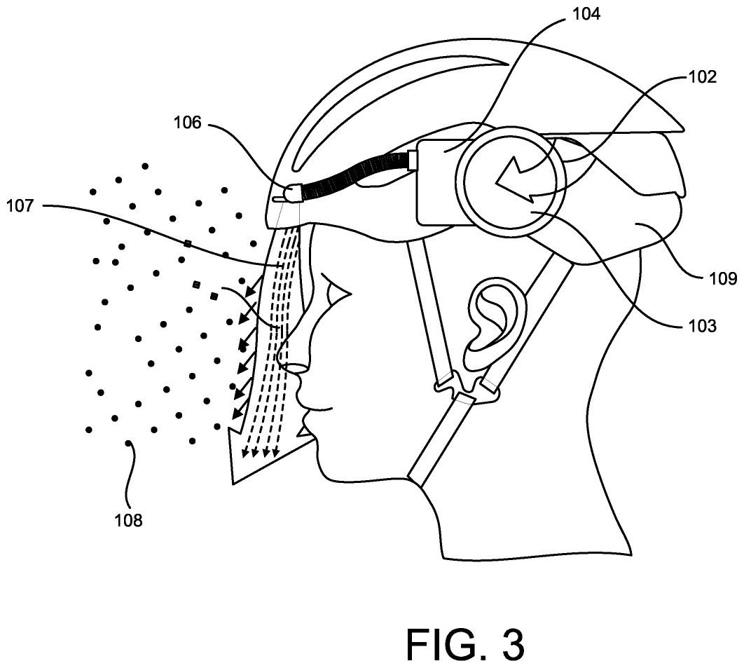

FIG. 4 is a schematic diagram according to an exemplary embodiment of the present disclosure.

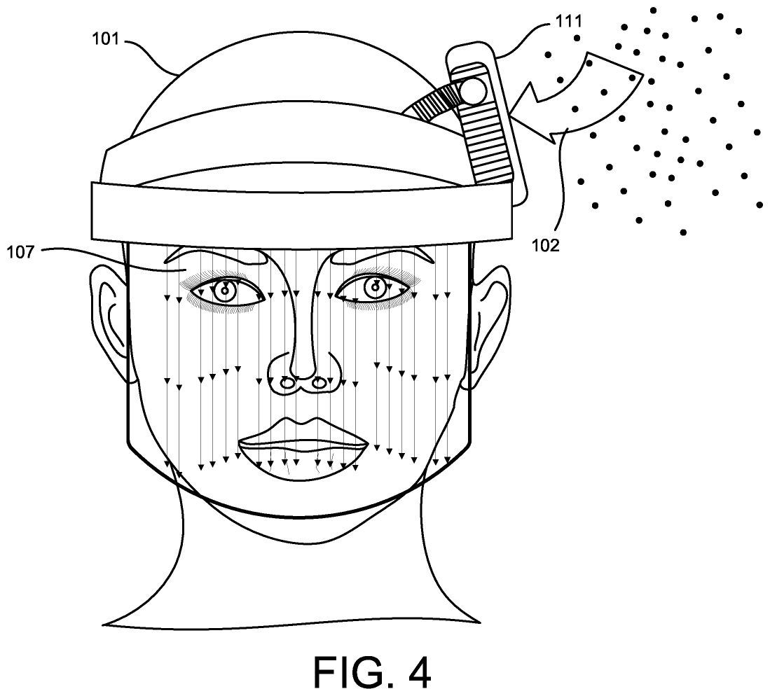

What is claimed is:

1) A facial mask apparatus and system comprising:
a headgear or headwear equipped with,
an air purifier, and
an air blower using a fan,
wherein the air purifier is configured to purify ambient air and the fan is configured to blow the purified ambient air in a downward direction from a top of a head area of a user towards a chin of the user and adjacent to a face of the user.

2) The face mask apparatus and system of claim 1, wherein the air purifier comprises a replaceable H13, HEPA filter and an activated carbon filter.

3) The face mask apparatus and system of claim 1, wherein the air purifier is capable of filtering ambient air to 99.97% purity.

4) The face mask apparatus and system of claim 1, wherein the air purifier is capable of filtering particles down to a diameter of 0.3 micron.

5) The apparatus and system of claim 1, wherein the apparatus weighs about one pound.

6) The face mask apparatus and system of claim 1, wherein a speed of air produced by the air blower is approximately 38 to 76 inches per second at a point of discharge.

7) The face mask apparatus and system of claim 1, wherein the fan is a centrifugal fan.

8) The face mask apparatus and system of claim 6, wherein the air blower has capacity of 17 GPM to maximum 30 GPM, with 3 speed or variable speed control.

9) The face mask apparatus and system of claim 1, wherein the fan, a battery, and a control system are mounted over a side, back or top of a hat or sun visors and an air hose is included to transfer air to a front of the user's face.

10) The face mask apparatus and system of claim 1, wherein an adjustable air nozzle is configured to be positioned right over a forehead of the user and is configured to pass air parallel with the face of the user to provide full coverage of the user's face, and the air nozzle is configured to evenly distributed air at a discharge point.

11) The face mask apparatus and system of claim 1, wherein the air purifier and the air blower are mounted as a kit over the headgear or headwear, which is a hat, cap, visor, helmet or hard hat, or the air purifier and air blower are built into the headgear or headwear.

12) The apparatus and system of claim 1, wherein a battery for the air blower is rechargeable and capable to provide required power to the system for a minimum of 6 hours of operation.

13) The face mask apparatus and system of claim 1, wherein a control system for the air blower has a battery level indicator to show an approximately remaining charge of battery.

14) The apparatus and system of claim 1, wherein the system is configured to be monitored/control through a mobile phone application with the user mobile phone.

15) The apparatus and system of claim 1, wherein the system has a short face shield to protect the user in harsh environments.
